(19) United States
(12) Patent Application Publication
(10) Pub. No.: US 2022/0183936 A1
(43) Pub. Date: Jun. 16, 2022

(54) Stable Skin Care Emulsion and Methods of Using the Same

(71) Applicant: The Procter & Gamble Company, Cincinnati, OH (US)

(72) Inventors: Matthew Clair EHRMAN, Singapore (SG); XiPing NG, Singapore (SG)

(21) Appl. No.: 17/549,989

(22) Filed: Dec. 14, 2021

Related U.S. Application Data
(60) Provisional application No. 63/125,021, filed on Dec. 14, 2020.

(51) Int. Cl. A61K 8/06; A61K 8/34; A61K 8/60; A61Q 19/00

(52) U.S. Cl. CPC A61K 8/062; A61K 8/342; A61K 2800/594; A61Q 19/00; A61K 8/60

(57) ABSTRACT

Sucrose esters such as sucrose laurate and sucrose dilaurate are notoriously difficult to emulsify in an oil-in-water emulsion and tend to destabilize these types of emulsions. It has now been found that combining a sucrose ester with certain hydrophobically modified aqueous rheology modifier and non-ionic stearic acid derivative emulsifiers can greatly improve emulsion stability.

### FIELD

The present disclosure is directed generally to improving the stability of an emulsion that contains a destabilizing ingredient. More specifically, the present disclosure is directed to improving the stability of a skin care composition emulsion that contains an emulsion destabilizing sucrose ester.

### BACKGROUND

Skin is the first line of defense against environmental insults that would otherwise damage sensitive underlying tissue and organs. For example, skin maintains a relatively water-impermeable barrier between an organism and its environment to prevent dehydration. Additionally, skin plays a key role in a person's physical appearance. Generally, most people desire to have younger, healthy looking skin. And to some of these people, the tell-tale signs of skin aging such as thinning skin, wrinkles, and age spots are an undesirable reminder of the disappearance of youth. As a result, treating the signs of aging in skin has become a booming business in youth-conscious societies. Treatments range from cosmetic creams and moisturizers to various forms of cosmetic surgery.

Numerous skin care agents, both natural and synthetic, are known for use in skin care compositions marketed to treat various skin conditions, especially those associated with aging. For example, U.S. Pat. No. 9,949,917 and U.S. Publication No. 2005/0220726 disclose cosmetic compositions containing sucrose fatty acid esters, such as sucrose laurate, sucrose dilaurate and sucrose trilaurate for use as skin lightening agents. However, when skin care compositions are provided in the form of an emulsion, these sucrose fatty acid esters may destabilize the emulsion due to their emulsifying properties (i.e., due to the hydrophilic and lipophilic moieties in these compounds). Emulsion instability may manifest in a variety of ways (e.g., flocculation, creaming, sedimentation, or coalescence), all of which are generally undesirable in a skin care composition.

Accordingly, it would be desirable to provide a skin care composition in the form of an emulsion that includes a sucrose fatty acid ester as a skin care agent and has desirable stability.

### SUMMARY

Disclosed herein is a skin care composition with improved emulsion stability, comprising: about 0.0001% to about 10% by weight of a sucrose ester selected from sucrose laurate, sucrose dilaurate, sucrose trilaurate and combinations thereof; about 0.01% to about 5% by weight of a hydrophobically modified aqueous rheology modifier; about 0.005% to about 5% by weight of a non-ionic, stearic acid derivative emulsifier; and a dermatologically acceptable carrier in the form of an oil-in-water emulsion. Also disclosed are methods of making and using the novel composition herein.

### BRIEF DESCRIPTION OF THE DRAWINGS

FIG. 1 illustrates an example of an unstable O/W emulsion exhibiting sedimentation.

FIG. 2 illustrates an example of a stable O/W emulsion.

FIG. 3 shows the instability index values for various emulsions.

### DETAILED DESCRIPTION

The use of sucrose fatty acid esters in skin care compositions is known. However, at least some sucrose fatty acid esters such as sucrose laurate and sucrose dilaurate may destabilize the oil phase of an emulsion, especially an oil-in-water emulsion. It has now been surprisingly discovered that selecting a specific emulsifier and rheology modifier can greatly improve the stability of an emulsion that contains a sucrose fatty acid ester. In particular, selecting an hydrophobically modified aqueous rheology modifier and a suitable non-ionic, a medium to long chain fatty-acid derivative emulsifier (e.g., a stearic acid-derived emulsifier) can greatly improve emulsion stability when a sucrose ester is present.

Reference herein to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive and combinable to form narrower ranges not explicitly disclosed. For example, delineated upper and lower range limits are interchangeable to create further ranges.

The compositions of the present invention can comprise, consist essentially of, or consist of, the essential components as well as optional ingredients described herein. As used herein, “consisting essentially of” means that the composition or component may only include additional ingredients that do not materially alter the basic and novel characteristics of the claimed composition or method. As used in the description and the appended claims, the singular forms “a”, “an”, and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Agent” refers to a material, as well any component thereof, intended to provide a particular benefit or function. For example, an emollient agent is a material intended to provide an emolliency benefit to skin (e.g., a fatty alcohol), and a thickening agent is a material generally intended to increase the viscosity of a composition.

“Apply” or “application”, as used in reference to a composition, means to apply or spread the compositions herein onto a bodily surface such as skin or hair.

“Cosmetic composition” means a composition that contains a cosmetic agent and is intended for non-therapeutic (i.e., non-medical) use. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Derivative,” herein, means amide, ether, ester, amino, carboxyl, acetyl, and/or alcohol derivatives of a given compound.

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein.

“Emulsion stability,” and variations thereof, refer to the ability of an emulsion to resist change in its properties over time. The changes may be physical or chemical and may visible or invisible. For example, a lack of emulsion stability may manifest as a visible phase separation (i.e., creaming or sedimentation). In another example, emulsion instability may manifest as an invisible (to the human eye) coalescence of the droplets in the dispersed phase that results in a change in viscosity or flow properties. Emulsion stability is characterized herein as Instability Index, which can be determined according to the Photo Centrifuge Test described in more detail below.

“Skin care” means regulating and/or improving a skin condition (e.g., skin health, appearance, or texture/feel). Some nonlimiting examples of improving a skin condition include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves a skin condition.

“Treatment period,” as used herein, means the length of time and/or frequency that a material or composition is applied to a target skin surface.

Composition

The compositions herein are in the form of an emulsion (e.g., oil-in-water emulsion) that contains a sucrose ester, an hydrophobically modified aqueous rheology modifier, a non-ionic medium to long chain fatty-acid derivative emulsifier, and a dermatologically acceptable carrier in the form of an oil-in-water emulsion. The combination of rheology modifiers and sucrose ester is selected to provide the emulsion with improved stability. The stable emulsions herein have an Instability Index of less than 5% (e.g., less than 4%, 3%, 2%, 1%, or even less than 0.5%). The stable emulsions herein have a relative instability of less than 25% (e.g., less than 20%, 15%, or even less than 10%) when normalized against a negative control. Stable emulsions can include very stable emulsions and partially stable emulsions, which may both be acceptable to consumers. A suitable method of determining Instability Index is described in more detail below.

The compositions herein may optionally include one or more additional skin actives or other ingredients of the type commonly included in topical skin care compositions. For example, the compositions herein may include a fatty alcohol (e.g., hexyldecanol) to act as a skin softening agent or emollient agent. When the composition includes a fatty alcohol, the combination of sucrose ester and fatty alcohol may be selected to provide a synergistic improvement in cellular ATP production, for example, as described in co-pending U.S. Provisional Ser. No. 63/125,011, filed on Dec. 14, 2020 by Hakozaki, et al., and titled “Method of Treating Oxidative Stress in Skin and Compositions Therefor.”

The skin care compositions herein may be made using conventional methods. However, in some instances, it may be difficult to solubilize the sucrose ester in the composition using conventional methods. In these instances, it may be desirable to solubilize the sucrose ester in 2-step dilution process using a glycol premix or other suitable solubilizing agent, which is then added to the composition. An example of this process is described in co-pending U.S. Provisional Ser. No. 63/124,870, filed on Dec. 14, 2020 by Tanaka, et al., and titled “Cosmetic Compositions Comprising Sucrose Esters and Solvents.”

The compositions herein may have a surfactant system that has an HLB of less than 10, in contrast with similar types of skin compositions that are known to have a surfactant system with an HLB of 12 or more (e.g., greater than 13, 14, or 15). In comparative compositions, when the HLB of the surfactant system falls below 10, the composition (i.e., emulsion) can become unstable. However, as shown in the examples below, merely adjusting HLB values may not overcome the instability observed when a sucrose ester is present in the composition. Thus, it can be important to specifically select a suitable combination of ingredients to ensure emulsion stability.

The composition herein may be a cosmetic composition, pharmaceutical composition, or cosmeceutical composition suitable for use on keratinous tissue (e.g., skin, hair, and nails), and may be provided in various product forms, including, but not limited to, solutions, suspensions, lotions, creams, gels, toners, sticks, sprays, aerosols, ointments, cleansing liquid washes and solid bars, pastes, foams, mousses, shaving creams, wipes, strips, patches, electrically-powered patches, hydrogels, film-forming products, facial and skin masks (with and without insoluble sheet), make-up such as foundations, eye liners, and eye shadows, and the like. In some instances, the composition form may follow from the particular dermatologically acceptable carrier chosen (i.e., an oil-in-water (O/W) emulsion). It may be particularly desirable to provide the present composition as a skin cream, lotion, serum, or essence.

Sucrose Ester

The compositions herein include an effective amount of an ester of sucrose and a fatty acid, wherein the fatty acid is selected from those with 12 to 24 carbon atoms (e.g., 12 to 22 carbon atoms or 12 to 18 carbon atoms). Particularly suitable fatty acids may be selected from those with saturated alkyl groups. In some instances, the sucrose ester is selected from the group consisting of sucrose laurate, sucrose dilaurate, sucrose trilaurate, derivatives of these, and combinations thereof. As used herein, “sucrose laurate” means a compound having the formula C₂₄H₄₄O₁₂ and CAS #25339-99-5; “sucrose dilaurate” means a compound having the formula C₃₆H₆₆O₁₃ and CAS #25915-57-5; and “sucrose trilaurate” means a compound having the formula C₄₄H₈₈O₁₄ and CAS #94031-23-9. The sucrose ester may be present at 0.0001% to 15% (e.g., 0.0002% to 10%, 0.001% to 15%, 0.025% to 10%, 0.05% to 7%, 0.05% to 5%, or even 0.1% to 3%) by weight of the composition.

In some instances, the sucrose ester may be a blend of two or more sucrose esters, wherein the two or more sucrose esters are present at a ratio of any one sucrose ester to another of 1:10 to 1:1 (e.g., 1:7, 1:5, 1:3, or 1:2). In some instances, the sucrose ester may be a blend of sucrose laurate and sucrose dilaurate, wherein sucrose laurate is present at 50% to 80%, by weight of the sucrose ester, and the sucrose dilaurate is present at 20% to 45%, by weight of the sucrose ester. Alternatively, the sucrose ester may be a blend of sucrose laurate, sucrose dilaurate and sucrose trilaurate, wherein sucrose dilaurate is present at 35% or more, by weight of the sucrose ester. A particularly suitable example of a sucrose ester for use herein is BC10034 from BASF®, which is a blend of sucrose laurate and sucrose dilaurate. The BC10034 sucrose ester material can have a ratio of sucrose laurate to sucrose dilaurate ranging from 3:1 to 3:2.

Dermatologically Acceptable Carrier

The compositions disclosed herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight.

The carrier herein is in the form of an oil-in-water emulsion. That is, the emulsion has a continuous aqueous phase and a dispersed oil phase. The oil phase may include silicone oils, non-silicone oils such as hydrocarbon oils, esters, ethers, and mixtures thereof. The aqueous phase may include water and/or water-soluble or water-miscible ingredients (e.g., ethanol, polyols such as glycerin, moisturizing agents, conditioning agents, anti-microbials, humectants and/or other skin care actives). The O/W emulsion may provide a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable, hydrophilic diluents. As used herein, “diluent” includes materials in which a material such as a sucrose ester can be dispersed, dissolved, or otherwise incorporated. Hydrophilic diluents include water, organic hydrophilic diluents such as lower monovalent alcohols e.g., C₁-C₄) and low molecular weight glycols and polyols, including propylene glycol, polyethylene glycol (e.g., molecular weight of 200 to 600 g/mole), polypropylene glycol (e.g., molecular weight of 425 to 2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

Rheology Modifiers

The compositions herein include 0.05% to 5% of a hydrophobically modified aqueous rheology modifier (e.g., thickening agent) to provide the composition with suitable rheological, stability, and skin feel properties. Some non-limiting examples of rheology modifier that may be suitable for use herein include crosslinked polyacrylate polymers, such as PEMULEN EZ-4U, PEMULEN TR-1, PEMULEN TR-2, and certain ULTREZ brand acrylates/C₁₀-30 alkyl acrylate crosspolymers, all available from Lubrizol®. Other examples include acrylates vinyl isodecanoate crosspolymer such as STABYLEN 30 sold by 3V Sigma®, polyacrylate crosspolymer-6 such as SEPIMAX ZEN sold by Seppic®, and sodium polyacryloyldimethyl taurate such as ARISTOFLEX SILK sold by Clariant. In some instances, it may be desirable to exclude polyacrylamide-based thickeners, such as SEPIGEL 305 brand polyacrylamide thickener, as these types of thickeners may destabilize the emulsion.

The rheology modifier of the composition herein may include a medium to long chain fatty-acid derivative emulsifier (e.g., about 12-20 carbon chain, alternatively 16-20 carbon chain). In some examples, the emulsifier can may include a non-ionic, medium to long chain fatty-acid derivative emulsifier, such as steareth-2 steareth-21, PEG-100 stearate, glycereth-25 pyrrolidonecarboxylic acid isostearate, and combinations of these. “Steric acid-derived emulsifier” refers to an emulsifier in which at least one of the lipophilic domains of the surfactant is comprised of a saturated 18-carbon chain (similar to stearic acid). These emulsifiers typically contain stearate, steareth, or isostearate in their chemical names and are often derived from stearic acid combined with other chemical moieties. Particularly suitable emulsifiers include stearic-acid derived emulsifiers with a hydrophilic-lipophilic balance (HLB) of 14 or more. The emulsifier may be present in the composition at 0.05% to 5% (e.g., 0.1% to 4%, 0.5% to 3% or even 1% to 2%). In some instances, it may be desirable to exclude certain polyether modified silicone emulsifiers, such a PEG-11 methyl ether dimethicone, PEG-12 dimethicone, PEG/PPG 19/19 dimethicone, or other PEGylated dimethicones, which may destabilize an oil-in-water emulsion.

Optional Ingredients.

The composition herein may include one or more optional ingredients known for use in topical skin care compositions, provided the optional components do not unacceptably alter the desired benefits of the present composition. In particular, the optional ingredients should not introduce undesirable instability to the emulsion. For example, it may be desirable to select optional ingredients that do not form complexes with other ingredients in the composition, especially pH sensitive ingredients like vitamin B3 compounds, salicylates and peptides. When optional skin care actives are included in the present compositions, it may be desirable to select skin care actives that function via different biological pathways from other skin actives present in the composition so that the actives do not interfere with one another.

The optional ingredients, when included, should be suitable for use in contact with human skin tissue without undue toxicity, incompatibility, instability, allergic response, and the like. The optional components, when present, may be included at an amount of about 0.001% to 50% (e.g., 0.01% to 40%, 0.1% to 30%, 0.5% to 20%, or 1% to 10%), by weight of the composition. Some nonlimiting examples of additional ingredients include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunscreen agents, sunless tanning agents, lubricants, anti-acne agents, anti-cellulite agents, chelating agents, anti-wrinkle actives, anti-atrophy actives, phytosterols and/or plant hormones, N-acyl amino acid compounds, antimicrobials, and antifungals. Some particularly suitable examples of additional ingredient include one or more skin care actives selected from the group consisting of vitamin B3 compounds (e.g., niacinamide), n-acyl amino acids (e.g., undecylenoyl phenylalanine), vitamin E compounds (e.g., tocopheryl acetate), palmitoylated dipeptides (e.g., palmitoyl-lysine-threonine), palmitoylated pentapeptides (e.g., palmitoyl-lysine-threonine-threonine-lysine-serine), vitamin A compounds (e.g., retinol and retinyl propionate), and combinations thereof. Other non-limiting examples of optional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/0092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

In some instances, it may be desirable to include a fatty alcohol in the skin care compositions. At least some fatty alcohols function as emollients, which help moisturize skin. Additionally or alternatively, some fatty alcohols such as hexyldecanol may improve penetration of certain skin actives (e.g., vitamin B3 compounds) into the skin and/or independently provide a skin health or appearance benefit. Fatty alcohols are high-molecular-weight, straight-chain primary alcohols that have the general structure:

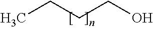

where n=8 to 32.

The fatty alcohol may be natural or synthetic, saturated or unsaturated, branched or straight-chain. Some nonlimiting examples of fatty alcohols commonly used in skin care compositions include caprylic, capryl, lauryl, myristyl, cetyl, stearyl, and behenyl alcohols. The fatty alcohols herein may be referred to generically by the number of carbon atoms in the molecule. For example, a “C12 alcohol” refers to an alcohol that has 12 carbon atoms in its chain (i.e., dodecanol). Some particularly suitable fatty alcohols for use herein include ricinoleates, 12-hydroxystearate, and hexyldecanol. The fatty alcohol may be included in the compositions herein at 0.0001% to 15% (e.g., 0.0002% to 10%, 0.001% to 15%, 0.025% to 10%, 0.05% to 7%, 0.05% to 5%, or even 0.1% to 3%) by weight of the composition.

Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof. Particularly suitable examples of conditioning agents include volatile or non-volatile silicone fluids such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization. Other examples of silicone fluids that may be suitable for use as conditioning agents are described in U.S. Pat. No. 5,011,681.

Method of Use

The present method includes identifying a target portion of keratinous tissue (e.g., skin or hair) on a person where treatment is desired and applying an effective amount of the composition thereto over the course of a treatment period. The target portion of keratinous tissue may be a facial skin surface, such as the forehead, perioral, chin, periorbital, nose, and/or cheek) or another part of the body (e.g., hands, arms, legs, back, chest). A target portion of skin where treatment is desired may be one that exhibits signs of oxidative stress or aging, such as fine lines, wrinkles, hyperpigmentation, uneven skin tone, and/or other visible skin features typically associated with aging. In some instances, the target portion of skin may not exhibit a visible sign of skin aging, but a user (e.g., a relatively young user) may still wish to target that area of skin, if it is one that typically develops such issues as a person ages. In this way, the present method may be used as a preventative measure to delay the onset of visible signs of skin aging.

A skin care composition herein may be applied to a target portion of skin and, if desired, to the surrounding skin at least once a day, twice a day, or on a more frequent daily basis, during the treatment period. When applied twice daily, the first and second applications are separated by at least 1 to 12 hours. Typically, the composition is applied in the morning and/or in the evening before bed. When used according to the methods herein, the present compositions may improve the appearance of skin affected by oxidative stress, for example, by increasing ATP production in skin cells.

A treatment period herein is ideally of sufficient time for the combination of sucrose ester and fatty alcohol present in the composition to increase ATP production in skin cells experiencing oxidative stress, thereby improving the appearance of visible signs of skin aging. The ATP production benefit provided by the present method can be demonstrated by a synergistic increase in ATP production relative to the use of the sucrose ester and fatty alcohol individually. In some instances, the present method may provide a synergistic increase of ATP production of at least 5% (e.g., 10%, 15%, 20%, 25%, or more) relative to the expected increase in ATP production. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a wrinkle or line) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied broadly to one or more skin surfaces. In certain embodiments, the compositions herein may be used as part of a multi-step beauty regimen, wherein the present composition may be applied before and/or after one or more other compositions.

Photo Centrifuge Test

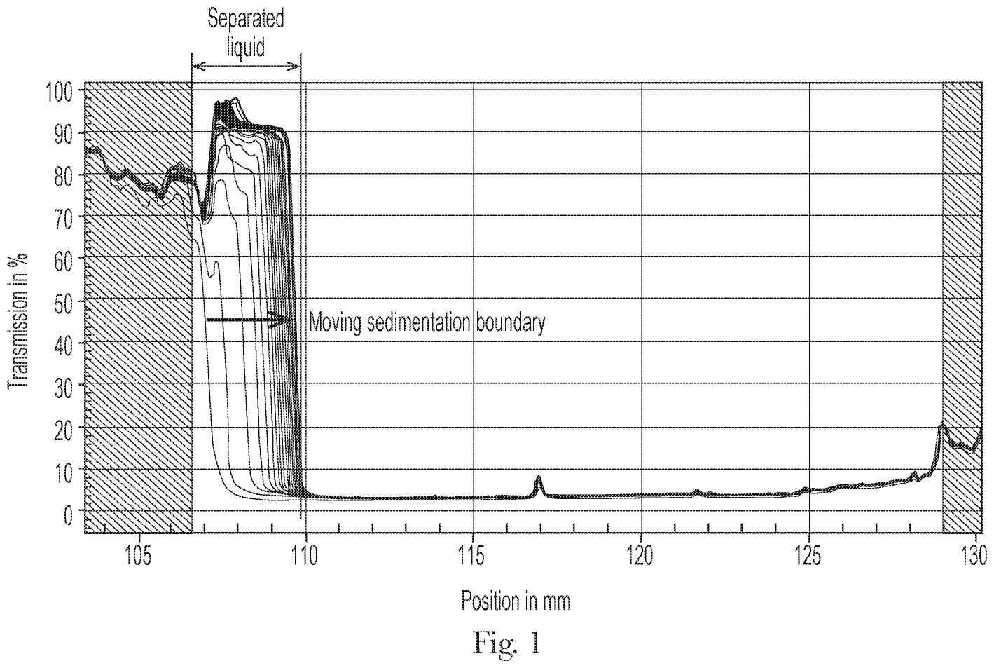

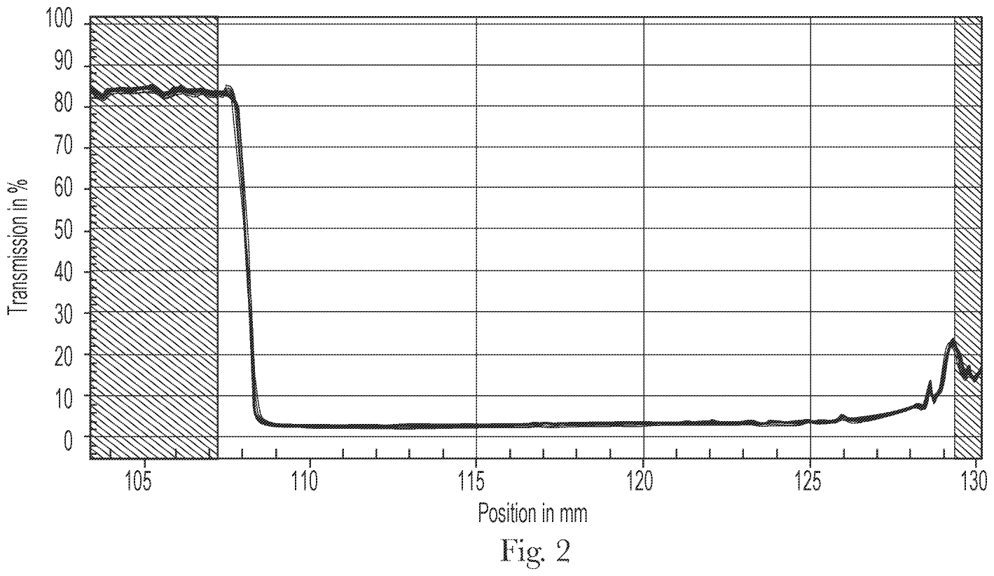

This test method provides a way to evaluate the emulsion stability of a sample using a dispersion analyzer (e.g., a LUMiSizer™ brand dispersion analyzer from LUM GMBH) at baseline and elevated temperature aging over time. The samples are placed in a suitable container for testing (e.g., LUM 2 mm, polycarbonate rectangular synthetic cell cuvettes (LUM GMBH: 110-131xx)) and run at 40° C. and 4000 rpm for 1 hour. Accelerated stability testing using photo centrifuge techniques is well documented in the literature (for example, reference: Badolato, G. G., et al. “Evaluation of long-term stability of model emulsions by multi-sample analytical centrifugation.” Surface and interfacial forces—from fundamentals to applications. Springer, Berlin, Heidelberg, 2008. 66-73). The method involves centrifugation of samples with illumination of parallel near-infrared or blue light over the entire sample cell. The transmitted light is detected by a CCD sensor and converted into extinction profiles. Changes in light transmission during centrifugation are indicative of multiple instability mechanisms such as sedimentation, creaming, flocculation, and/or emulsion coalescence. For emulsions, both sedimentation and creaming can be observed by increasing transmission at either the top of the sample (sedimentation) or bottom of the sample (creaming) based on relative densities of the phase separation. For example, FIG. 1 illustrates an O/W emulsion exhibiting sedimentation, which is observed as lower density oil separating from the rest of the emulsion under centrifugation at the top of the sample. In contrast, FIG. 2 illustrates an example of a stable O/W emulsion.

The instability of an emulsion measured via photo centrifugation can be quantified with an instability index (Detloff, T., T. Sobisch, and D. Lerche. “Instability index.” Dispersion Letters Technical 4 (2013): 1-4.). The instability index is based on an increase in light transmission, also known as clarification, due to either sedimentation or creaming. The index is a dimensionless number between 0 and 1, where ‘0’ indicates completely stable with no change during centrifugation and ‘1’ is completely unstable as indicated by a change in light transmission over the region of interest. The dimensionless index can be multiplied by 100% to represent the instability index as a percentage. The index provides a relative ranking of stability for different samples tested. The measured instability index may be normalized against a negative control to provide a more informative indication of emulsion stability.

The instability index can be calculated using a suitable software tool (e.g., SEPView® software or equivalent) according to the equations below. Equation 1 is used to calculate clarification (difference between first and subsequent transmission). Equation 2 is used to calculate clarification change up to a certain time for profile i, which is calculated as the summation of the incremental clarifications within the region of interest. Equation 3 is used to calculate the maximum clarification possible. Equation 4 calculates the instability index as the change in transmission divided by the maximum change possible within the region of interest.

Tᵢ^{diff}=Tᵢ−T₁ for i≥2 Equation 1:
where: T is transmission and i is the profile number

$$Δ {T}_{i} = {\sum }_{{r}_{min}}^{{r}_{max}} {T}_{i}^{diff} \qquad Equation 2$$

where: r is the region of interest position max and min value and j is the position increment (14 microns in LUMiSizer™)

ΔTₘₐₓ=(T̅_{End}−T̅₁)·(jᵣ_{ₘₐₓ}−jᵣ_{ₘᵢₙ}) Equation 3:
where T̅_{End} is the mean transmission for a cell with water only and T̅_{1_} is the first transmission profile and j are the position increments at min and max r positions.

$$\frac{Δ {T}_{i}}{Δ {T}_{max}} = \frac{{\sum }_{{r}_{min}}^{{r}_{max}} {T}_{i}^{diff}}{( {\overline{T}}_{E n d} - {\overline{T}}_{1} ) \cdot ( {j}_{{r}_{max}} - {j}_{{r}_{min}} )} \cdot 100 \% \qquad Equation 4$$

As can be seen from the equations, the instability index is a relative ranking based on the region interest vs. the maximum clarification possible in that region. For the instability index measurements reported herein, the region of interest was taken over the entire sample cell, since the absolute value of the instability index is affected by the selected region of interest. As a result, relative comparisons for a given region of interest may be of more interest than the absolute values reported. Relative comparisons may be provided by normalizing the observed instability index against a control.

In addition to the instability index, the rate of change of the instability index is indicative of the separation kinetics. This can be evaluated by plotting the instability index vs. time. This rate of change is related to the velocity of the sedimentation or creaming which can also be quantified based on Stokes Law. As noted, the sedimentation velocity is defined by Stokes Law as shown in equation 5. Where v is the velocity, ρ is the density, r is the particle size, η is the dynamic viscosity of the continuous phase, g is the gravity, a is the particle concentration, and RCA is the relative centrifugal acceleration.

$$v = \frac{2}{9} \cdot \frac{Δρ \cdot {r}^{2}}{η} \cdot g \cdot f ( α ) \cdot RCA \qquad Equation 5$$

It is believed, without being limited by theory, that, the primary driver in emulsion instability is the change in the droplet radius, r, from sucrose ester-driven coalescence, which correlates to a higher sedimentation velocity.

### EXAMPLES AND COMBINATIONS

### Example 1: Formulations

Table 1 below provides examples of stable emulsion skin care compositions containing a sucrose ester. The exemplary compositions can be made as follows. Prepare the sucrose ester premix by combining the sucrose ester, pentylene glycol and water and mixing until the sucrose ester is fully dissolved. Heat may be used as appropriate to aid in dissolving the sucrose ester. Separately, the water phase is prepared by dispersing the thickener(s) and polymeric emulsifier(s) in the aqueous phase. After the thickener(s) is dispersed and homogenous, add any additional emulsifier(s) (e.g., high-HLB emulsifier) to the water phase. Add the remaining aqueous phase ingredients and adjust the pH as desired (e.g., pH 5-7). In a separate container, combine the oil phase ingredients and mix until homogenous, with heating as necessary (e.g., to melt any solid materials). Next emulsify the oil phase into the water phase by slowly adding the oil phase while mixing, continue mixing until the emulsion is fully homogeneous. After the emulsion is fully homogeneous, add any remaining ingredients while mixing until homogeneous. The sucrose ester pre-mix phase can be added to the batch before or after the emulsification step and then mixing until the batch is homogeneous.

TABLE 1
Exemplary formulations

| OIL PHASE | Wt % | | | | | | | | | |
|---|---|---|---|---|---|---|---|---|---|---|
| | I | II | III | IV | V | VI | VII | VIII | IX | X |
| Hexyldecanol | 5 | 5 | 5 | 5 | 5 | 5 | 5 | 5 | 5 | 1.25 |
| Phytosteryl/octyldodecyl | 1 | 1 | 1 | 1 | 1 | 1 | 1 | 1 | 1 | 1 |
| lauroyl glutamate¹ | | | | | | | | | | |
| Tocopheryl acetate | 0.5 | 0.5 | 0.5 | 0.5 | 0.5 | 0.5 | 0.5 | 0.5 | 0.5 | 0.5 |
| Dimethicone/vinyl | 1 | 1 | 1 | 1 | 1 | 1 | 1 | 1 | 1 | 1 |
| dimethicone crosspolymer² | | | | | | | | | | |
| PEG-11 methyl ether dimethicone³ | 0.25 | 0.25 | 0.25 | — | 0.25 | 0.25 | 0.25 | 0.25 | 0.25 | 0.25 |
| Polysorbate 20 | — | — | — | — | — | — | — | — | 0.5 | 0.5 |
| Glycereth-25 PCA isostearate | 1 | — | 1 | 1 | 1 | 1 | 1 | 1 | 1 | 0.25 |
| Stereath-2 | 0.05 | 0.1 | — | 0.05 | 0.05 | 0.05 | 0.05 | 0.05 | 0.1 | 0.025 |
| Stereath-21 | 0.45 | 0.9 | — | 0.45 | 0.45 | 0.45 | 0.45 | 0.45 | 0.9 | 0.225 |
| PEG-100 stearate | — | — | 1 | — | — | — | — | — | — | — |
| SUCROSE ESTER PRE-MIX | | | | | | | | | | |
| Water | 3 | 3 | 3 | 3 | 3 | 3 | 3 | 3 | 3 | 3 |
| Pentylene glycol | 2 | 2 | 2 | 2 | 2 | 2 | 2 | 2 | 2 | 2 |
| Sucrose ester⁴ | 1 | 1 | 1 | 1 | 1 | 1 | 1 | 1 | 1 | 1 |
| WATER PHASE | | | | | | | | | | |
| Water | QS | QS | QS | QS | QS | QS | QS | QS | QS | QS |
| Acrylates/c10-30 alkyl | 0.1 | 0.1 | 0.1 | 0.1 | 0.24 | 0.1 | 0.24 | — | 0.32 | 0.32 |
| acrylate crosspolymer⁵ | | | | | | | | | | |
| Acrylates/c10-30 alkyl | 0.40 | 0.40 | 0.40 | 0.40 | 0.20 | — | — | — | — | — |
| acrylate crosspolymer⁶ | | | | | | | | | | |
| Acrylates/c10-30 alkyl | — | — | — | — | — | 0.40 | 0.20 | — | — | — |
| acrylate crosspolymer⁷ | | | | | | | | | | |
| Acrylates vinyl | — | — | — | — | — | — | — | 0.40 | — | — |
| isodeconate crosspolymer⁸ | | | | | | | | | | |
| Xanthan gum | — | — | — | — | — | — | — | — | 0.05 | 0.05 |
| Polyacrylamide (and) C13-14 | — | — | — | — | — | — | — | — | 0.6 | 0.6 |
| isoparaffin (and) laureth-7⁹ | | | | | | | | | | |
| Xylitol | 1.5 | 1.5 | 1.5 | 1.5 | 1.5 | 1.5 | 1.5 | 1.5 | 1.5 | 1.5 |
| Niacinamide | 5 | 5 | 5 | 5 | 5 | 5 | 5 | 5 | 5 | 5 |
| Sodium benzoate | 0.05 | 0.05 | 0.05 | 0.05 | 0.05 | 0.05 | 0.05 | 0.05 | 0.05 | 0.05 |
| Disodium EDTA | 0.1 | 0.1 | 0.1 | 0.1 | 0.1 | 0.1 | 0.1 | 0.1 | 0.1 | 0.1 |
| Phenoxyethanol | 0.25 | 0.25 | 0.25 | 0.25 | 0.25 | 0.25 | 0.25 | 0.25 | 0.25 | 0.25 |
| Benzyl alcohol | 0.2 | 0.2 | 0.2 | 0.2 | 0.2 | 0.2 | 0.2 | 0.2 | 0.2 | 0.2 |
| D-panthenol | 1 | 1 | 1 | 1 | 1 | 1 | 1 | 1 | 1 | 1 |
| Aminomethyl propanol⁺ | 0.4 | 0.4 | 0.4 | 0.4 | 0.4 | 0.4 | 0.4 | 0.4 | 0.23 | 0.23 |
| Undecylenoyl phenylalanine¹⁰ | 0.2 | 0.2 | 0.2 | 0.2 | 0.2 | 0.2 | 0.2 | 0.2 | 0.2 | 0.2 |
| Pentylene glycol | 1 | 1 | 1 | 1 | 1 | 1 | 1 | 1 | 1 | 1 |
| Glycerin | 2.5 | 2.5 | 2.5 | 2.5 | 2.5 | 2.5 | 2.5 | 2.5 | 2.5 | 2.5 |
| Butylene glycol | 2 | 2 | 2 | 2 | 2 | 2 | 2 | 2 | 2 | 2 |

| | XI | XII | XIII |
|---|---|---|---|
| Hexyldecanol | 5 | 5 | 5 |
| Phytosteryl/octyldodecyl | 1 | 1 | 1 |
| lauroyl glutamate¹ | | | |
| Tocopheryl acetate | 0.5 | 0.5 | 0.5 |
| Dimethicone/vinyl | 1 | 1 | 1 |
| dimethicone crosspolymer² | | | |
| PEG-11 methyl ether dimethicone³ | 0.25 | 0.25 | — |
| Polysorbate 20 | | | |
| Glycereth-25 PCA isostearate | — | — | — |
| Stereath-2 | 0.1 | 0.1 | — |
| Stereath-21 | 0.9 | 0.9 | — |
| PEG-100 stearate | — | — | 1 |
| SUCROSE ESTER PRE-MIX | | | |
| Water | 3 | 3 | 3 |
| Pentylene glycol | 2 | 2 | 2 |
| Sucrose ester⁴ | 1 | 1 | 1 |
| WATER PHASE | | | |
| Water | QS | QS | QS |
| Acrylates/c10-30 alkyl | 0.24 | 0.24 | 0.24 |
| acrylate crosspolymer⁵ | | | |
| Acrylates/c10-30 alkyl | — | — | — |
| acrylate crosspolymer⁶ | | | |
| Acrylates/c10-30 alkyl | — | — | — |
| acrylate crosspolymer⁷ | | | |
| Acrylates vinyl | — | — | — |
| isodeconate crosspolymer⁸ | | | |
| Xanthan gum | — | — | — |
| Polyacrylamide (and) C13-14 | — | — | — |
| isoparaffin (and) laureth-7⁹ | | | |
| Polyacrylate crosspolymer-6¹² | 0.4 | — | — |
| Sodium polyacryloyldimethyl taurate¹³ | — | 0.4 | 0.4 |
| Xylitol | 1.5 | 1.5 | 1.5 |
| Niacinamide | 5 | 5 | 5 |
| Sodium benzoate | 0.05 | 0.05 | 0.05 |
| Disodium EDTA | 0.1 | 0.1 | 0.1 |
| Phenoxyethanol | 0.25 | 0.25 | 0.25 |
| Benzyl alcohol | 0.2 | 0.2 | 0.2 |
| D-panthenol | 1 | 1 | 1 |
| Aminomethyl propanol⁺ | 0.4 | 0.4 | 0.4 |
| Undecylenoyl phenylalanine¹⁰ | 0.2 | 0.2 | 0.2 |
| Pentylene glycol | 1 | 1 | 1 |
| Glycerin | 2.5 | 2.5 | 2.5 |
| Butylene glycol | 2 | 2 | 2 |

¹ELDEW PS-203 available from Ajinomoto ®
²KSG-16 available from Shin-Etsu ®
³KF-6011 available from Shin-Etsu ®
⁴BC10034 from BASF ®
⁵CARBOPOL ULTREZ20 available from Lubrizol ®
⁶PEMULEN TR-1 available from Lubrizol ®
⁷PEMULEN EZ-4U available from Lubrizol ®
⁸STABLYEN 30 available from 3V Sigma ®
⁹SEPIGEL 305 available from Seppic ®
¹⁰SEPIWHITE available from Seppic ®
11. DRYFLO TS available from Nouryon ®
¹²SEPIMAX ZEN available from Seppic ®
¹³ARISTOFLEX SILK available from Clariant ®
⁺Balance to approximate neutral pH 5-7

### Example 2—Instability Index Variability

This example demonstrates the effect of various emulsifiers and rheology modifiers on Instability Index. In this example, the stability test samples were prepared as described in Example 1, and tested according to the Photo Centrifuge method described above. The instability index is quantified using Lum GMBH SEPView® brand software by selecting the relevant region of interest at the air/liquid interface and the bottom of the sample for a full cell analysis. Instability index values for each test composition and control were determined after aging at 50° C. for 2 weeks. As previously noted, stable emulsions have an instability index of less than 5% (e.g., less than 4%, 3%, 2%, 1%, or even less than 0.5%). Partially stable emulsions are noted where minimal oil separation is observed and/or creaming is present in the sample after photo-centrifuge testing, typically observed in the range of 2.5%-5% instability index. Very stable emulsions are noted when there is no oil separation whatsoever is observed in the sample after photo-centrifuge testing, typically observed in the range less than 2.5%, alternatively less than 2%. Partially stable emulsions may be consumer acceptable, although they can be slightly less preferred as compared to very stable emulsions. The compositions tested are set forth in Table 2 below. Composition A, which does not include a non-ionic, stearic acid-derived emulsifier, is used as the negative control. The Instability Index results were normalized against the negative control.

TABLE 2
Test Formulas

| | A | B | C | D | E | F | G | H |
|---|---|---|---|---|---|---|---|---|
| OIL PHASE | Wt % | | | | | | | |
| Hexyldecanol | 5 | 1.25 | 0.11 | 5 | 5 | 5 | 5 | 5 |
| Phytosteryl/octyldodecyl | 1 | 1 | 1 | 1 | 1 | 1 | 1 | 1 |
| lauroyl glutamate¹ | | | | | | | | |
| Tocopheryl acetate | 0.5 | 0.5 | 0.5 | 0.5 | 0.5 | 0.5 | 0.5 | 0.5 |
| Dimethicone/vinyl | 1 | 1 | 1 | 1 | 1 | 1 | 1 | 1 |
| dimethicone crosspolymer² | | | | | | | | |
| PEG-11 methyl ether dimethicone³ | 0.25 | 0.25 | 0.25 | 0.25 | 0.25 | 0.25 | 0.25 | 0.25 |
| Glycereth-25 PCA isostearate | — | — | — | 1 | — | — | 1 | 1 |
| Stereath-2 | — | — | — | — | 0.05 | 0.1 | 0.1 | 0.05 |
| Stereath-21 | — | — | — | — | 0.45 | 0.9 | 0.9 | 0.45 |
| SUCROSE ESTER PRE-MIX | | | | | | | | |
| Water | 3 | 3 | 3 | 3 | 3 | 3 | 3 | 3 |
| Pentylene glycol | 2 | 2 | 2 | 2 | 2 | 2 | 2 | 2 |
| Sucrose dilaurate⁴ | 1 | 1 | 1 | 1 | 1 | 1 | 1 | 1 |
| WATER PHASE | | | | | | | | |
| Water | QS | QS | QS | QS | QS | QS | QS | QS |
| Acrylates/c10-30 alkyl | 0.28 | 0.28 | 0.28 | 0.28 | 0.28 | 0.28 | 0.28 | 0.24 |
| acrylate crosspolymer⁵ | | | | | | | | |
| Acrylates/c10-30 alkyl | — | — | — | — | — | — | — | 0.40 |
| acrylate crosspolymer⁶ | | | | | | | | |
| Polyacrylamide (and) C13-14 | 0.6 | 0.6 | 0.6 | 0.6 | 0.6 | 0.6 | 0.6 | — |
| isoparaffin (and) laureth-7⁷ | | | | | | | | |
| Xylitol | 1.5 | 1.5 | 1.5 | 1.5 | 1.5 | 1.5 | 1.5 | 1.5 |
| Niacinamide | 5 | 5 | 5 | 5 | 5 | 5 | 5 | 5 |
| Sodium benzoate | 0.05 | 0.05 | 0.05 | 0.05 | 0.05 | 0.05 | 0.05 | 0.05 |
| Disodium EDTA | 0.1 | 0.1 | 0.1 | 0.1 | 0.1 | 0.1 | 0.1 | 0.1 |
| Phenoxyethanol | 0.25 | 0.25 | 0.25 | 0.25 | 0.25 | 0.25 | 0.25 | 0.25 |
| Benzyl alcohol | 0.2 | 0.2 | 0.2 | 0.2 | 0.2 | 0.2 | 0.2 | 0.2 |
| D-panthenol | 1 | 1 | 1 | 1 | 1 | 1 | 1 | 1 |
| pH ADJUSTMENT | | | | | | | | |
| Aminomethyl propanol⁺ | 0.189 | 0.189 | 0.189 | 0.189 | 0.189 | 0.189 | 0.189 | 0.42 |
| Undecylenoyl phenylalanine⁸ | 0.2 | 0.2 | 0.2 | 0.2 | 0.2 | 0.2 | 0.2 | 0.2 |
| Water | 2 | 2 | 2 | 2 | 2 | 2 | 2 | 2 |

RESULTS

| | | | | | | | | |
|---|---|---|---|---|---|---|---|---|
| 2W@50° C. Instability Index | 14.4% | 8.13% | 1.63% | 20.67% | 7.83% | 2.73% | 4.70% | 0.4% |
| Normalized vs. Neg. Control | 100% | 56% | 11% | 144% | 54% | 19% | 33% | 3% |

⁺Balance to pH of 5.5-6.5.
¹ELDEW PS-203 available from Ajinomoto ®
²KSG-16 available from Shin-Etsu ®
³KF-6011 available from Shin-Etsu ®
⁴BC10034 available from BASF ®
⁵CARBOPOL ULTREZ 20 available from Lubrizol ®
⁶PEMULEN TR-1 available from Lubrizol ®
⁷SEPIGEL 305 available from Seppic ®
⁸SEPIWHITE available from Seppic ®

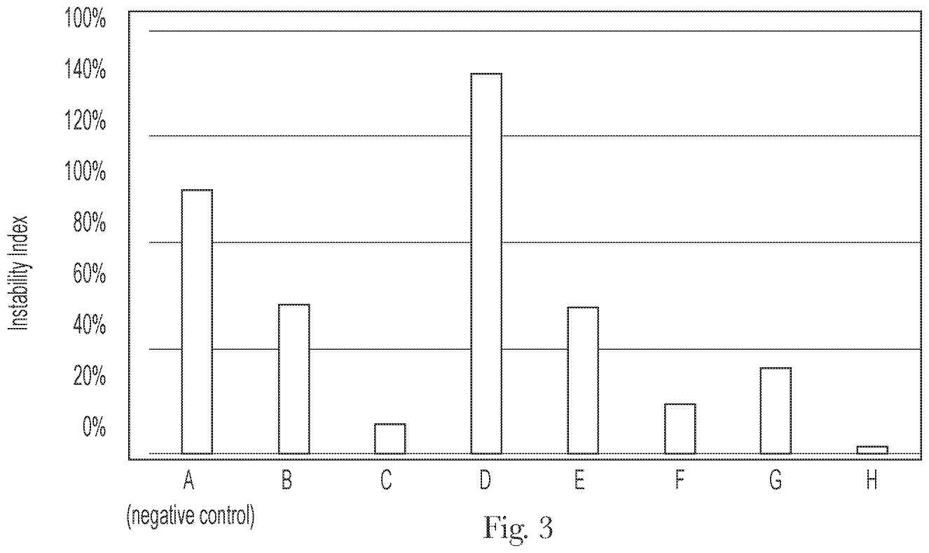

The results of the test are summarized below in Table 3 and illustrated in FIG. 3. As can be seen in Table 3 and FIG. 3, the inventive composition H, which includes a suitable combination of sucrose ester, hydrophobically modified polymeric thickener (e.g., Pemulen™) and non-ionic, stearic acid-derived emulsifier, was the most stable composition versus comparative examples. It exhibits an Instability Index of less than 1% and a relative Instability Index of less than 5% when normalized against the negative control.

Example C was also stable and has a lower level of hexyldecanol, as compared to A and B, in combination with sucrose dilaurate.

Partially stable formulations were observed by using non-ionic, stearic acid-derived emulsifier in the presence of a reduced hydrophobically modified polymeric thickener Carbopol Ultrez 20™ which is not as effective as the increased hydrophically modified polymer Pemulen TR-1™.

TABLE 3
Instability Index

| | (A) | (B) | (C) | (D) | (E) | (F) | (G) | (H) |
|---|---|---|---|---|---|---|---|---|
| 2W@ 50° C. | 14.4% | 8.13% | 1.63% | 20.67% | 7.83% | 2.73% | 4.70% | 0.4% |
| Instability Index | | | | | | | | |
| Normalized vs. | 100% | 56% | 11% | 144% | 54% | 19% | 33% | 3% |
| Neg Control | | | | | | | | |
| Observations | Unstable; | Unstable; | Stable | Unstable; | Unstable; | Partially | Partially | Stable |
| | oil | oil | | oil | oil | Stable; | Stable; | |
| | separation | separation | | separation | separation | creaming | creaming | |

### Example 3—Sucrose Ester Destabilizes O/W Emulsions

The example demonstrates the instability effect that a sucrose ester can have on an O/W emulsion skin care composition by comparing a test composition containing sucrose dilaurate to a control composition that does not contain sucrose dilaurate. The test composition and the control composition were both O/W emulsions and were identical in all respects, except that the test composition contains 1% sucrose dilaurate (BC10034 from BASK)). The compositions were made according to the method described in Example 1 and tested according to the Photo Centrifuge Test described above. Samples were prepared in LUM 2 mm, polycarbonate rectangular synthetic cell and run on a LUMISIZER brand dispersion analyzer at a constant temperature of 40° C. at 4000RPM for 1 hour with measurement sampling every 10 seconds. Analysis was conducted using Lum GMBH SEPView® software to quantify the instability index by selecting the relevant region of interest for the total sample cell. Instability index values for each composition were determined after aging at an elevated temperature of 50° C. for 2 weeks similar to the previous example. The results of the test are summarized below in Table 4. As can be seen in Table 4, the addition of the sucrose ester to the O/W emulsion composition introduced significant instability relative to the control.

TABLE 4

| | A | B |
|---|---|---|
| OIL PHASE | Wt % | |
| Hexyldecanol | 5 | 5 |
| Phytosteryl/octyldodecyl lauroyl | 1 | 1 |
| glutamate¹ | | |
| Tocopheryl acetate | 0.5 | 0.5 |
| Dimethicone/vinyl dimethicone | 1 | 1 |
| crosspolymer² | | |
| PEG-11 methyl ether dimethicone³ | 0.25 | 0.25 |
| SUCROSE ESTER PRE-MIX | | |
| Water | 3 | 3 |
| Pentylene glycol | 2 | 2 |
| Sucrose dilaurate⁴ | 1 | — |
| WATER PHASE | | |
| Water | QS | QS |
| Acrylates/c10-30 alkyl acrylate | 0.28 | 0.28 |
| crosspolymer⁵ | | |
| Polyacrylamide (and) C13-14 isoparaffin | 0.6 | 0.6 |
| (and) laureth-7⁶ | | |
| Xylitol | 1.5 | 1.5 |
| Niacinamide | 5 | 5 |
| Sodium benzoate | 0.05 | 0.05 |
| Disodium EDTA | 0.1 | 0.1 |
| Phenoxyethanol | 0.25 | 0.25 |
| Benzyl alcohol | 0.2 | 0.2 |
| D-panthenol | 1 | 1 |
| Butylene glycol | 2 | 2 |
| Pentylene glycol | 1 | 1 |
| Glycerin | 2.5 | 2.5 |
| pH ADJUSTMENT | | |
| Aminomethyl propanol⁺ | 0.189 | 0.189 |
| Undecylenoyl phenylalanine⁷ | 0.2 | 0.2 |
| Water | 2 | 2 |

RESULTS

| | | |
|---|---|---|
| 2 W @50° C. Instability Index | 14.4% | 0.3% |
| Normalized vs. Neg. Control | 100% | 2% |

⁺Balance to pH of 5.5-6.5.
¹ELDEW PS-203 available from Ajinomoto ®
²KSG-16 available from Shin-Etsu ®
³KF-6011 available from Shin-Etsu ®
⁴BC10034 available from BASF ®
⁵CARBOPOL ULTREZ 20 available from Lubrizol ®
⁶SEPIGEL 305 available from Seppic ®
⁷SEPIWHITE available from Seppic ®

### Example 4—Criticality of Rheology Modifier and Emulsifier Selection

This example demonstrates the importance of selecting a suitable rheology modifier and emulsifier to overcome the emulsion instability caused by a sucrose ester. A dose response of polysorbate-20, which is a well-known ethoxylated sorbital ester emulsifier, was conducted to rebalance the HLB with sucrose dilaurate present. It was found that simply rebalancing the HLB of the emulsifiers with polysorbate-20 was insufficient to stabilize the oil in water emulsion even at a polysorbate-20 level of 10% as shown in Table 5 and 6. It was also found that using a hydrophobically modified rheology modifier alone such as Pemulen TR-1™ was also not sufficient to fully stabilize the emulsion. Surprisingly, it was found that a combination of a hydrophobically modified rheology modifier (e.g., Pemulen™, Stabylen30™, Sepimax Zen™, Aristoflex Silk™) and stearic acid derivative emulsifier (e.g., PEG-100 stearate, stereath-2/stereath-21 blends, and/or glycerth-25 PCA isostearate) was able to suitably stabilize the emulsion, and a dose response improvement of increasing stability is observed when combining the hydrophobically modified rheology modifier with a stearic acid derivative emulsifier in certain combinations as shown in Table 7.

The HLB experiment compositions are shown in Table 5. The base formula was modified by varying the levels of polysorbate-20. Test samples of each composition were prepared according to the method described in Example 1 and tested according to the Photo Centrifuge Test method described above. The base composition is free of sucrose ester, while the test compositions include 1% sucrose ester and varying amounts of polysorbate-20. The results of the test are summarized in Table 6.

TABLE 5

| | A | B | C | D |
|---|---|---|---|---|
| OIL PHASE | Wt % | | | |
| Hexyldecanol | 5 | 5 | 5 | 5 |
| Phytosteryl/octyldodecyl lauroyl | 1 | 1 | 1 | 1 |
| glutamate¹ | | | | |
| Tocopheryl acetate | 0.5 | 0.5 | 0.5 | 0.5 |
| Dimethicone/vinyl dimethicone | 1 | 1 | 1 | 1 |
| crosspolymer² | | | | |
| PEG-11 methyl ether dimethicone³ | 0.25 | 0.25 | 0.25 | 0.25 |
| Polysorbate 20 | — | 0.5 | 2 | 10 |
| SUCROSE ESTER PRE-MIX* | | | | |
| Water | 3 | 3 | 3 | 3 |
| Pentylene glycol | 2 | 2 | 2 | 2 |
| Sucrose dilaurate⁴ | — | 1 | 1 | 1 |
| WATER PHASE | | | | |
| Water | QS | QS | QS | QS |
| Acrylates/c10-30 alkyl acrylate | 0.28 | 0.28 | 0.28 | 0.28 |
| crosspolymer⁵ | | | | |
| Polyacrylamide (and) C13-14 | 0.6 | 0.6 | 0.6 | 0.6 |
| isoparaffin (and) laureth-7⁶ | | | | |
| Xylitol | 1.5 | 1.5 | 1.5 | 1.5 |
| Niacinamide | 5 | 5 | 5 | 5 |
| Sodium benzoate | 0.05 | 0.05 | 0.05 | 0.05 |
| Disodium EDTA | 0.1 | 0.1 | 0.1 | 0.1 |
| Phenoxyethanol | 0.25 | 0.25 | 0.25 | 0.25 |
| Benzyl alcohol | 0.2 | 0.2 | 0.2 | 0.2 |
| D-panthenol | 1 | 1 | 1 | 1 |
| Butylene glycol | 2 | 2 | 2 | 2 |
| Pentylene glycol | 1 | 1 | 1 | 1 |
| Glycerin | 2.5 | 2.5 | 2.5 | 2.5 |
| pH ADJUSTMENT | | | | |
| Aminomethyl propanol⁺ | 0.189 | 0.189 | 0.189 | 0.189 |
| Undecylenoyl phenylalanine⁷ | 0.2 | 0.2 | 0.2 | 0.2 |
| Water | 2 | 2 | 2 | 2 |

RESULTS

| | | | | |
|---|---|---|---|---|
| 2 W @50° C. Instability Index | 0.3% | 20.1% | 23% | 52.97% |
| Normalized vs. Neg. Control | 2% | 140% | 160% | 368% |

⁺Balance to pH of 5.5-6.5.
*Sucrose Ester (SE) premix only used for test compositions.
¹ELDEW PS-203 available from Ajinomoto ®
²KSG-16 available from Shin-Etsu ®
³KF-6011 available from Shin-Etsu ®
⁴BC10034 available from BASF ®
⁵CARBOPOL ULTREZ 20 available from Lubrizol ®
⁶SEPIGEL 305 available from Seppic ®
⁷SEPIWHITE available from Seppic ®

TABLE 6

| | Instability Index | Observation |
|---|---|---|
| Base formula (0% SE) | 0.3% | Stable |
| Base formula + 1% SE + 0.5% | 20.1% | Oil Phase separation |
| Polysorbate | | |
| Base formula + 1% SE + 2% | 23% | Oil Phase separation |
| Polysorbate | | |
| Base formula + 1% SE + 10% | 52.97% | Oil Phase separation |
| Polysorbate | | |

Tables 7A, 7B, and 7C provide additional inventive and comparative example compositions, which demonstrate the criticality of selecting the proper combination of sucrose ester, hydrophobically modified aqueous rheology modifier, and non-ionic stearic acid-derived emulsifier. Composition A is used as a positive control and composition B is used as a negative control. The examples compositions in Tables 7A-7C were made using the methods described herein.

TABLE 7A

| INGREDIENTS | A | B | C | D | E | F |
|---|---|---|---|---|---|---|
| Water | QS | QS | QS | QS | QS | QS |
| Sucrose ester¹ | — | 1 | 1 | 1 | 1 | 1 |
| Acrylates/C10-30 Alkyl | — | — | — | — | 0.20 | 0.20 |
| Acrylate Crosspolymer² | | | | | | |
| Acrylates/C10-30 Alkyl | 0.28 | 0.28 | 0.28 | 0.28 | 0.24 | 0.24 |
| Acrylate Crosspolymer³ | | | | | | |
| Polyacrylamide (and) C13-14 | 0.6 | 0.6 | 0.6 | 0.6 | — | — |
| Isoparaffin (and) Laureth-74⁴ | | | | | | |
| Glycereth-25 PCA isostearate | — | — | — | — | — | — |
| PEG-100 stearate | — | — | 0.5 | 1 | — | 0.5 |
| Steareth-2 | — | — | — | — | — | — |
| Steareth-21 | — | — | — | — | — | — |
| Hexyldecanol | 5 | 5 | 5 | 5 | 5 | 5 |
| Dimethicone/Vinyl Dimethicone | 1 | 1 | 1 | 1 | 1 | 1 |
| Crosspolymer (KSG-16) ⁵ | | | | | | |
| PEG-11 Methyl Ether | 0.25 | 0.25 | 0.25 | 0.25 | 0.25 | 0.25 |
| Dimethicone (KF6011) ⁶ | | | | | | |
| Phytosteryl/Octyldodecyl Lauroyl | 1 | 1 | 1 | 1 | 1 | 1 |
| Glutamate (Eldew PS-203) | | | | | | |
| Tocopheryl Acetate | 0.5 | 0.5 | 0.5 | 0.5 | 0.5 | 0.5 |
| Inositol | 0.3 | 0.3 | 0.3 | 0.3 | 0.3 | 0.3 |
| Xylitol | 1.5 | 1.5 | 1.5 | 1.5 | 1.5 | 1.5 |
| Niacinamide | 5 | 5 | 5 | 5 | 5 | 5 |
| Sodium Benzoate | 0.05 | 0.05 | 0.05 | 0.05 | 0.05 | 0.05 |
| Disodium EDTA | 0.1 | 0.1 | 0.1 | 0.1 | 0.1 | 0.1 |
| Phenoxyethanol | 0.25 | 0.25 | 0.25 | 0.25 | 0.25 | 0.25 |
| Benzyl Alcohol | 0.2 | 0.2 | 0.2 | 0.2 | 0.2 | 0.2 |
| D-Panthenol | 1 | 1 | 1 | 1 | 1 | 1 |
| Pentylene Glycol | 3 | 3 | 3 | 3 | 3 | 3 |
| Aminomethyl Propanol⁺ | 0.189 | 0.189 | 0.189 | 0.189 | 0.27 | 0.27 |
| Undecylenoyl Phenylalanine | 0.2 | 0.2 | 0.2 | 0.2 | 0.2 | 0.2 |
| (Sepiwhite MSH) | | | | | | |
| Glycerin | 2.5 | 2.5 | 2.5 | 2.5 | 2.5 | 2.5 |
| Butylene Glycol | 2 | 2 | 2 | 2 | 2 | 2 |

RESULTS

| | | | | | | |
|---|---|---|---|---|---|---|
| 2W@50° C. Instability Index | 0.3% | 14.4% | 2.13% | 4.8% | 6.83% | 1.23% |
| Normalized Instability Index | 2% | 100% | 15% | 33% | 47% | 9% |

TABLE 7B

| Ingredients | G | H | I | J | K | L | M |
|---|---|---|---|---|---|---|---|
| Water | QS | QS | QS | QS | QS | QS | QS |
| Sucrose ester¹ | 1 | 1 | 1 | 1 | 1 | 1 | 1 |
| Acrylates/C10-30 Alkyl | 0.20 | 0.20 | 0.40 | 0.40 | 0.40 | 0.40 | 0.40 |
| Acrylate Crosspolymer² | | | | | | | |
| Acrylates/C10-30 Alkyl | 0.24 | 0.24 | 0.24 | 0.24 | 0.24 | 0.24 | 0.24 |
| Acrylate Crosspolymer³ | | | | | | | |
| Glycereth-25 PCA isostearate | — | — | — | — | — | 1.0 | 1.0 |
| PEG-100 stearate | 1 | 1.5 | 0.50 | 1 | 1.5 | — | — |
| Steareth-2 | — | — | — | — | — | 0.10 | 0.05 |
| Steareth-21 | — | — | — | — | — | 0.90 | 0.45 |
| Hexyldecanol | 5 | 5 | 5 | 5 | 5 | 5 | 5 |
| Dimethicone/Vinyl Dimethicone | 1 | 1 | 1 | 1 | 1 | 1 | 1 |
| Crosspolymer (KSG-16) | | | | | | | |
| PEG-11 Methyl Ether | 0.25 | 0.25 | 0.25 | 0.25 | 0.25 | 0.25 | 0.25 |
| Dimethicone (KF6011) ⁶ | | | | | | | |
| Phytosteryl/Octyldodecyl | 1 | 1 | 1 | 1 | 1 | 1 | 1 |
| Lauroyl Glutamate | | | | | | | |
| (Eldew PS-203) | | | | | | | |
| Tocopheryl Acetate | 0.5 | 0.5 | 0.5 | 0.5 | 0.5 | 0.5 | 0.5 |
| Inositol | 0.3 | 0.3 | 0.3 | 0.3 | 0.3 | 0.3 | 0.3 |
| Xylitol | 1.5 | 1.5 | 1.5 | 1.5 | 1.5 | 1.5 | 1.5 |
| Niacinamide | 5 | 5 | 5 | 5 | 5 | 5 | 5 |
| Sodium Benzoate | 0.05 | 0.05 | 0.05 | 0.05 | 0.05 | 0.05 | 0.05 |
| Disodium EDTA | 0.1 | 0.1 | 0.1 | 0.1 | 0.1 | 0.1 | 0.1 |
| Phenoxyethanol | 0.25 | 0.25 | 0.25 | 0.25 | 0.25 | 0.25 | 0.25 |
| Benzyl Alcohol | 0.2 | 0.2 | 0.2 | 0.2 | 0.2 | 0.2 | 0.2 |
| D-Panthenol | 1 | 1 | 1 | 1 | 1 | 1 | 1 |
| Pentylene Glycol | 3 | 3 | 3 | 3 | 3 | 3 | 3 |
| Aminomethyl Propanol⁺ | 0.27 | 0.27 | 0.42 | 0.42 | 0.42 | 0.42 | 0.42 |
| Undecylenoyl Phenylalanine | 0.2 | 0.2 | 0.2 | 0.2 | 0.2 | 0.2 | 0.2 |
| (Sepiwhite MSH) | | | | | | | |
| Glycerin | 2.5 | 2.5 | 2.5 | 2.5 | 2.5 | 2.5 | 2.5 |
| Butylene Glycol | 2 | 2 | 2 | 2 | 2 | 2 | 2 |

RESULTS

| | | | | | | | |
|---|---|---|---|---|---|---|---|
| 2W@50° C. Instability Index | 0.73% | 0.7% | 0.6% | 0.57% | 0.67% | 0.47% | 0.4% |
| Normalized Instability Index | 5% | 5% | 4% | 4% | 5% | 3% | 3% |

TABLE 7C

| Ingredients | N | O | P | Q | R | S |
|---|---|---|---|---|---|---|
| Water | QS | QS | QS | QS | QS | QS |
| Sucrose ester¹ | 1 | 1 | 1 | 1 | 1 | 1 |
| Acrylates/C10-30 Alkyl | 0.20 | 0.40 | 0.40 | — | — | — |
| Acrylate Crosspolymer² | | | | | | |
| Acrylates/C10-30 Alkyl | 0.24 | 0.24 | 0.24 | 0.24 | 0.24 | 0.24 |
| Acrylate Crosspolymer³ | | | | | | |
| Acrylates vinyl isodeconate | — | — | — | 0.4 | — | — |
| crosspolymer⁷ | | | | | | |
| Polyacrylate Crosspolymer-6⁸ | — | — | — | — | 0.4 | — |
| Sodium Polyacryloyldimethyl | — | — | — | — | — | 0.4 |
| Taurate⁹ | | | | | | |
| Glycereth-25 PCA isostearate | 1.00 | 1.00 | 1.00 | 1.00 | 1.00 | 1.00 |
| PEG-100 stearate | — | 0.5 | 1.00 | — | — | — |
| Steareth-2 | 0.10 | — | — | 0.1 | 0.1 | 0.1 |
| Steareth-21 | 0.90 | — | — | 0.9 | 0.9 | 0.9 |
| Hexyldecanol | 5 | 5 | 5 | 5 | 5 | 5 |
| Dimethicone/Vinyl Dimethicone | 1 | 1 | 1 | 1 | 1 | 1 |
| Crosspolymer (KSG-16) | | | | | | |
| PEG-11 Methyl Ether | 0.25 | 0.25 | 0.25 | 0.25 | 0.25 | 0.25 |
| Dimethicone (KF6011) ⁶ | | | | | | |
| Phytosteryl/Octyldodecyl Lauroyl | 1 | 1 | 1 | 1 | 1 | 1 |
| Glutamate (Eldew PS-203) | | | | | | |
| Tocopheryl Acetate | 0.5 | 0.5 | 0.5 | 0.5 | 0.5 | 0.5 |
| Inositol | 0.3 | 0.3 | 0.3 | 0.3 | 0.3 | 0.3 |
| Xylitol | 1.5 | 1.5 | 1.5 | 1.5 | 1.5 | 1.5 |
| Niacinamide | 5 | 5 | 5 | 5 | 5 | 5 |
| Sodium Benzoate | 0.05 | 0.05 | 0.05 | 0.05 | 0.05 | 0.05 |
| Disodium EDTA | 0.1 | 0.1 | 0.1 | 0.1 | 0.1 | 0.1 |
| Phenoxyethanol | 0.25 | 0.25 | 0.25 | 0.25 | 0.25 | 0.25 |
| Benzyl Alcohol | 0.2 | 0.2 | 0.2 | 0.2 | 0.2 | 0.2 |
| D-Panthenol | 1 | 1 | 1 | 1 | 1 | 1 |
| Pentylene Glycol | 3 | 3 | 3 | 3 | 3 | 3 |
| Aminomethyl Propanol⁺ | 0.27 | 0.42 | 0.42 | 0.63 | 0.204 | 0.204 |
| Undecylenoyl Phenylalanine | 0.2 | 0.2 | 0.2 | 0.2 | 0.2 | 0.2 |
| (Sepiwhite MSH) | | | | | | |
| Glycerin | 2.5 | 2.5 | 2.5 | 2.5 | 2.5 | 2.5 |
| Butylene Glycol | 2 | 2 | 2 | 2 | 2 | 2 |

RESULTS

| | | | | | | |
|---|---|---|---|---|---|---|
| 2W@50° C. Instability Index | 1.03% | 0.6% | 0.47% | 0.3% | 2.27% | 0.53% |
| Normalized Instability Index | 7% | 4% | 3% | 2% | 16% | 4% |

⁺Balance to pH of 5.5-6.5
¹BC10034 from BASF ®
²PEMULEN TR-1 from Lubrizol ®
³CARBOPOL ULTREZ 20 from Lubrizol ®
⁴SEPIGEL 305 from Seppic ®
5. KSG-16 available from Shin-Etsu ®
⁶ KF-6011 available from Shin-Etsu ®
⁷STABLYEN 30 available from 3V Sigma ®
⁸SEPIMAX ZEN from Seppic ®
⁹ARISTOFLEX SILK from Clariant

In addition to determining the Instability Index for compositions A-T, visual observations were also made to describe emulsion stability or instability. Table 7D below provides a summary of the visual observations made of each composition.

TABLE 7D

| Composition | Observation |
|---|---|
| A | Stable |
| B | Unstable-Complete oil phase separation |
| C | Partially Stable-Creaming |
| D | Partially Stable-Creaming with minor oil separation |
| E | Unstable-Complete oil phase separation |
| F | Stable |
| G | Stable |
| H | Stable |
| I | Stable |
| J | Stable |
| K | Stable |
| L | Stable |
| M | Stable |
| N | Stable |
| O | Stable |
| P | Stable |
| Q | Stable |
| R | Partially Stable-creaming |
| S | Stable |
| T | Stable |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition with improved emulsion stability, comprising:
a) about 0.0001% to about 10% by weight of a sucrose ester selected from sucrose laurate, sucrose dilaurate, sucrose trilaurate and combinations thereof;
b) about 0.01% to about 5% by weight of a hydrophobically modified aqueous rheology modifier;
c) about 0.005% to about 5% by weight of a non-ionic, medium to long chain fatty-acid derivative emulsifier; and
d) a dermatologically acceptable carrier in the form of an oil-in-water emulsion.

2. The skin care composition of claim 1, wherein the skin care composition exhibits an Instability Index value of less than about 2% according the Photo Centrifuge Test.

3. The skin care composition of claim 1, wherein the skin care composition exhibits a normalized Instability Index value of less than about 20%, relative to a negative control, according the Photo Centrifuge Test.

4. The skin care composition of claim 3, wherein the sucrose laurate is present at 50% to 80%, by weight of the sucrose ester, and the sucrose dilaurate is present at 20% to 45%, by weight of the sucrose ester.

5. The skin care composition of claim 1, wherein the medium to long chain fatty-acid derivative emulsifier is a non-ionic, stearic acid derivative emulsifier.

6. The skin care composition of claim 5, wherein the non-ionic, stearic acid derivative emulsifier is selected from stereath-2 stereath-21, PEG-100 stearate glycereth-25 pyrrolidonecarboxylic acid isostearate.

7. The skin care composition of claim 1, wherein the hydrophobically modified aqueous rheology modifier is selected from acrylates/C₁₀-30 alkyl acrylate crosspolymer, acrylates vinyl isodeconate crosspolymers, polyacrylate crosspolymer-6, sodium polyacryloyldimethyl taurate and combinations thereof.

8. The skin care composition of claim 1, wherein the composition is free of polyether-modified silicone emulsifiers.

9. The skin care composition of claim 1, further comprising about 0.0001% to about 10%, by weight of a fatty alcohol.

10. The skin care composition of claim 9, wherein the fatty alcohol is hexyldecanol.

11. The skin care composition of claim 1, further comprising an additional ingredient selected from vitamins, minerals, peptides, sugar amines, sunscreen agents, oil control agents, flavonoid compounds, anti-oxidants, preservatives, protease inhibitors, tyrosinase inhibitors, anti-inflammatory agents, moisturizing agents, exfoliating agents, skin lightening agents, lubricants, anti-acne actives, chelating agents, anti-wrinkle actives, anti-atrophy actives, phytosterols, N-acyl amino acid compounds, antimicrobials, and antifungals, conditioning agents, emulsifiers, rheology modifiers, and combinations of these.

12. The skin care composition of claim 11, wherein the additional ingredient is a skin care active selected from vitamin B3 compounds, undecylenoyl phenylalanine, vitamin E compounds, palmitoylated dipeptides, palmitoylated pentapeptides, vitamin A compounds, and combinations thereof.

13. The skin care composition of claim 11, wherein the composition further comprises an emulsifier selected from PEG-100 Stearate, Steareth-2, Steareth-21, and glycereth-25 pyrrolidonecarboxylic acid isostearate.

14. A method of cosmetically treating skin, comprising:
i. identifying a target portion of skin where treatment is desired; and
ii. applying a skin care composition to the target portion of skin, the skin care composition comprising:
1. about 0.0001% to about 10% of a sucrose ester selected from sucrose laurate, sucrose dilaurate, sucrose trilaurate, and combinations thereof;
2. about 0.01% to about 5% by weight of an hydrophobically modified aqueous rheology modifier;
3. about 0.005% to about 5% by weight of a non-ionic, stearic acid derivative emulsifier; and
4. a dermatologically acceptable carrier in the form of an oil-in-water emulsion wherein the composition improves at least one of the health and appearance of the target portion of skin over the course of a treatment period.

15. The method of claim 14, wherein the composition has an Instability Index of less than about 5%.

16. The method of claim 14, wherein the sucrose ester is a combination of sucrose laurate and sucrose dilaurate.

17. The method of claim 16, wherein the sucrose laurate is present at about 50% to about 80%, by weight of the sucrose ester, and the sucrose dilaurate is present at about 20% to about 45%, by weight of the sucrose ester.

18. The method of claim 14, wherein the hydrophobically modified aqueous rheology modifier is selected from acrylates/C₁₀-30 alkyl acrylate crosspolymer, acrylates vinyl isodeconate crosspolymers, polyacrylate crosspolymer-6, sodium polyacryloyldimethyl taurate and combinations thereof and combinations thereof.

19. The method of claim 14, wherein the composition is free of polyether-modified silicone emulsifiers.

20. The method of claim 14, further comprising about 0.0001% to about 10%, by weight of a fatty alcohol, wherein the fatty alcohol is hexyldecanol.
